(19) United States
(12) Patent Application Publication
(10) Pub. No.: US 2007/0269793 A1
(43) Pub. Date: Nov. 22, 2007

(54) Bioactive Boxes for Cellular Cultures

(76) Inventors: Marie-Danielle Nagel, Betheny (FR); Gilbert Legeay, Saint Saturnin (FR)

Correspondence Address: YOUNG & THOMPSON, 745 SOUTH 23RD STREET, 2ND FLOOR, ARLINGTON, VA, 22202, US

(21) Appl. No.: 10/580,679

(22) PCT Filed: Nov. 26, 2004
(86) PCT No.: PCT/FR04/03031

§ 371 Date: Jun. 6, 2007

(30) Foreign Application Priority Data
Nov. 28, 2003 (FR) 0313993

(51) Int. Cl. C12Q 1/00; C12M 3/00

(52) U.S. Cl. 435/4; 435/287.1

(57) ABSTRACT

The invention relates to bioactive boxes for cellular cultures comprising a bilayer which is arranged on the bottom of said boxes and provided with an internal primary layer made of hydroxypropylmethylcellulose, or polyvinyl alcohol (PVA) and an external bioactive layer made of carboxypropylmethylcellulose. Said invention also relates to the use of the inventive boxes for carrying out methods for studying cellular ageing, cellular differentiation and apoptosis, method s for screening anti-ageing molecules, methods for screening antitumoral molecules, methods for in vitro diagnosis of the antitumoral molecule malignancy and consequently methods for in vitro prognosticating tumors or for study methods related to researches on signalling controlling morphology, bioadhesion, cellular proliferation and intercellular communications.

A subject of the present invention is bioactive dishes for cell cultures, and their uses for implementing methods for studying cell ageing, cell differentiation, and apoptosis, methods for screening anti-ageing molecules, methods for screening anti-tumor molecules, methods for in vitro diagnosis of tumor-cell malignancy and therefore methods for in vitro prognosis of tumors, or study methods relating to research into signalling controlling morphology, bioadhesion, cell proliferation and intercellular communication.

The Inventors' works initially related to the use of cuprophan membranes which were plated on the bottom of Petri dishes, then they tested PVP (polyvinylpyrrolidone), HEC (hydroxyethylcellulose), HPMC (hydroxypropylmethylcellulose), and CMC (carboxymethylcellulose) coatings. The advantages and drawbacks of these different materials are summarized hereafter.

1) the cuprophan membrane

Main characteristics:
Stability: 48 hours
Aggregated (formation of cell aggregates) and/or rounded cell morphology
Suitable for studying cell signalling at 45, 90 and 180 minutes (Faucheux et al., 1998, 1999, 2000, 2001, and 2002, and Duval et al., 1999)
Reduced cell proliferation
Induces apoptosis (in particular on Swiss 3T3 fibroblasts)

Main drawbacks:
difficult to use (folds),
impossibility of industrial transfer,
no longer produced commercially.

2) PVP coating

Main characteristics:
Spread-out cell morphology (results identical to those obtained on the polystyrene (PS) control)

This coating is therefore unusable.

3) HEC coating

Main characteristics:
Stability 48 hours
Aggregated cell morphology
Slightly reduced cell proliferation

Main drawbacks:
difficult to use so as to have a regular deposit on the support

4) HPMC coating

Main characteristics:
Stability: 48 hours
Aggregated cell morphology
Reduced cell proliferation
Homogeneous, well-covering coating, and easy to use
Coating can be used for the injection of aggregated cells at 90 minutes (cuprophan unfavourable because of folds); e.g. study of the functionality of communicating junctions by the injection of lucifer yellow.

Its main drawback resides in the fact that it does not allow the activation of the cells beyond a few hours.

5) CMC coating

Main characteristics:
Stability: 48 hours or more
Aggregated and/or rounded cell morphology
Reduced cell proliferation.

Drawback: incomplete covering of the support: a PS ring remains accessible to the cells at the periphery (fairly difficult to use, cannot be used industrially).

The present invention results from the demonstration by the Inventors of the fact that the HPMC (or polyvinyl alcohol (PVA))-CMC bilayer makes it possible to remedy the drawbacks of the coatings studied above.

The advantages of the CMC or PVA bilayer on HPMC are the following:
Coating perfectly covering and easy to produce
Stability: at least 5 days
Sterilizable
Aggregated (Swiss 3T3, L929 fibroblasts, melanomas, osteoblast-like lines MC 3T3 E14 and MC 3T3 E1 24) or rounded (human fibroblasts) cell morphology
Reduced cell proliferation (B16C3, B16F0, B16F10 melanomas), MC 3T3, Swiss 3T3 and L929 murine fibroblasts.
Differentiation of the cells evidenced by melanin synthesis and increased tyrosinase activity for the 3 melanoma lines
Apoptosis induced at 24 and 48 hours: caspase 3 activity increased to 24 and 48 hours and caspase 9 activity increased to 6 hours for the Swiss 3T3 fibroblasts.

A subject of the present invention is bioactive dishes for cell cultures comprising on their bottom a bilayer comprising an internal primary layer made of hydroxypropylmethylcellulose (HPMC), or polyvinyl alcohol (PVA) in contact with the bottom of the dishes, and an external bioactive layer made of carboxypropylmethylcellulose situated on said internal layer.

The abovementioned bioactive dishes of the invention are further characterized in that they are presented in the form of Petri dishes, such as polystyrene Petri dishes of commercial origin, or in the form of multi-well plates, on the bottom of which the bilayer is situated.

Advantageously, the abovementioned bioactive dishes of the invention are characterized in that the thicknesses of the internal HPMC or PVA layer, and of the external CMC layer, are a few microns, in particular approximately 1 to 5 microns.

The invention also relates to a method for preparing bioactive dishes as defined above, characterized in that it comprises:

a stage of activation of the surface of the bottom of the dishes by electromagnetic discharges,

the depositing of the internal HPMC layer on the bottom of the dishes, then drying,

the depositing of the external bioactive layer on the dried primary layer obtained in the preceding stage, then drying.

A subject of the invention is also the use of bioactive dishes as defined above, for the implementation of:

methods for studying cell ageing, cell differentiation, and apoptosis,

methods for screening anti-ageing molecules intended to prevent and delay the effects of ageing,

methods for screening antitumor molecules intended for the treatment of cancer (research into a potentializing, or cumulative effect relative to the inducing effect of the coating),

methods for in vitro diagnosis of tumor-cell malignancy by measurement of the residual ability of cancer cells to differentiate, and to enter into apoptosis and therefore methods for in vitro tumor prognosis,

or study methods relating to research into signalling controlling morphology, bioadhesion, cell proliferation and intercellular communication.

A more particular subject of the invention is a method for studying cell ageing, cell differentiation, and apoptosis, characterized in that it comprises:

a stage of culturing cells to be studied in the dishes defined above,

the observation of the cells by microscope in order to study their morphology,

and/or the detection, or even the quantification, of cell differentiation, by measurement of cell proliferation, synthesized proteins, and specific membrane markers expressed,

and/or the detection, or even the quantification, of the apoptosis of cells, by measurement of viability (trypan blue exclusion test), activation of the caspases (fluorometric or calorimetric methods, western blotting), chromatin fragmentation (TUNEL test), or formation of apoptotic bodies (Hoechst staining).

A subject of the invention is also a method for screening anti-ageing molecules intended to prevent and delay the effects of ageing, characterized in that it comprises:

a stage of culturing cells, such as fibroblasts, in the presence of anti-ageing molecules to be studied, in the culture dishes defined above,

the observation of the cells by microscope in order to study their morphology,

and/or the detection, or even the quantification, of the proliferation, and syntheses, in particular of cell proteins such as collagen,

and comparison with the observations and results obtained on cultures of cells used as controls, said control cultures being carried out by culturing said cells in the absence of said anti-ageing molecules to be studied, in the dishes defined above.

The invention also relates to a method for screening antitumor molecules intended for the treatment of cancer, characterized in that it comprises:

a stage of culturing cells, such as animal or human melanoma cells, in the presence of the antitumor molecules to be studied, in the culture dishes defined above,

observation of the cells by microscope in order to study their morphology and their differentiation (melanogenesis),

and/or the detection, or even the quantification, of their proliferation, differentiation (detection of melanin synthesis and tyrosinase activity, fibronectin synthesis, expression of tumor markers such as MelCam, expression of cadherins, (detected by western blotting for example) and apoptosis (study of caspase activities by spectrofluorometry, western blotting for example and other methods such as TUNEL, Hoechst staining etc.)

and comparison with the observations and results obtained on cell cultures used as controls, said control cultures being carried out by culturing said cells in the absence of said antitumor molecules to be studied, in the culture dishes defined above.

A subject of the invention is also a method for in vitro diagnosis of the malignancy of tumor cells by measurement of the residual ability of cancer cells to differentiate, characterized in that it comprises:

a stage of culturing cancer cells, such as human melanoma cells obtained from biopsies, in the culture dishes defined above,

the observation of the cells by microscope in order to study their morphology and differentiation, and/or the detection, or even the quantification, of their proliferation (detection of intracellular melanin synthesis according to the technique described by De Pauw-Gillet et al. (Anticancer Research 1990, 10, 391-96), of tyrosinase activity (assay method used described by Steinberg et al. (J Cell. Physiol. 1976, 87, 265-76), of fibronectin, (immunocytochemistry, RT-PCR)), of their viability, and of apoptosis.

The invention also relates to the application of the abovementioned method of diagnosis to the in vitro prognosis of tumors.

A subject of the invention is also study methods relating to research into cell signalling (cAMP and MAPK pathways in particular), in relation to morphology, bioadhesion (relative to the state of activation or inactivation of the integrins), proliferation, intercellular communication (detection of cadherins, connexins, integrins), starting with various techniques such as microscopic observations, western blotting, RT-PCR, flow cytometry, immunolabellings and injection of lucifer yellow into the aggregates, said methods being carried out by culturing the cells to be studied in culture dishes defined above, observation and measurement of the abovementioned events.

The invention is further illustrated by means of the following detailed description of a method for obtaining a culture dish comprising a bilayer as defined above according to the invention, as well as their use within the framework of cell culture.

### I) EXPERIMENTAL DESCRIPTION OF THE PRODUCTION OF PETRI-DISH COATINGS

The operations are carried out with dishes of commercial origin, generally made of polystyrene. They can be small, average or large in size. They can also be multiple-well systems.

The operations were carried out with dishes obtained from Greiner® Bio-one.

The dishes are used as they are (no washing which is likely to pollute or alter the state of the surface).

The treatments are carried out in several stages which are carried out in a “clean room” environment.

1) Surface Activation by Electromagnetic Discharges, (Plasma, Corona Discharges).

For example an RF 13.56 MHz system is used per plasma, with a reactor of approximately 20 litres capacity, proceeding as follows:

The objects are placed inside the reactor, then a vacuum is applied (down to approximately 1 Pa), then gas is introduced, for example oxygen up to a partial pressure of 5 to 10 Pa is reached. The electromagnetic discharge is initiated and maintained (this is automatic) at a certain power (for example 50 W) and for 5 minutes.

At the end of this time the dishes are recovered. The effectiveness of the treatment is assessed by the measurement of wetting with the water drop test (the contact angle is less than 25°).

2) Depositing of a Layer (Primary)

In the minutes following the surface activation a layer of the polymer HPMC E4M is deposited from an aqueous solution, which has been degassed in order to drive off the dissolved air, at 0.2%/eppi. The dishes are filled then emptied after a few seconds, left to drain in aerated medium then dried in a ventilated oven at 50° C. for approximately 1 hour.

The effectiveness of the treatment is assessed by the measurement of wetting with the water drop test: the contact angle is of the order of 50 to 60°.

3) Depositing of a Bioactive Layer

The second layer of CMC 7LF polymer is deposited as previously from an aqueous solution (water ppi) previously degassed, and at a concentration of 0.2%.

After draining and drying at 50° C. the dishes are packaged either individually or in batches of 10 and under a flow of nitrogen.

For quality control, the effectiveness of the treatment is assessed by the measurement of wetting with the water drop test: the contact angle of is of the order of 40 to 45°.

Subsequently, the dishes thus obtained are in particular designated cellulose-coated dishes or Bioactive Cell-culture Dishes (BCDs).

### II) BIOLOGICAL ASPECTS

1) Preparation of the Cellulose-Coated Dishes

Before use, the cellulose-coated dishes are treated for 1 hour at laboratory temperature with a 1% antibiotic solution (penicillin, streptomycin) in ultra-pure water. They are then rinsed 3 times with ultra-pure water.

Recently, HPMC and CMC solutions have been filtered in a sterile manner before being used for the coating. The dishes were then only subjected to an ultra-pure water bath for 1 hour at laboratory temperature before being seeded with the cells.

After sterilization of the bioactive cell-culture dishes (BCDs) by ethylene oxide and desorption of the gas according to the slow or accelerated methods used as standard in the industry, the cell aggregates can be maintained in culture for a week if complete medium is added on the 3^{rd} or 4^{th} day of culture.

2) Biological Characterization of the Coating

After the usual treatment followed by incubation for 1 hour at 37° C. in the presence of a human or bovine fibronectin (Fn) solution at a concentration of 3 μg/cm² and 3 washings in ultra-pure water, the dishes are seeded with cells and the morphology of the latter is observed after 3 hours. The absence of cell spreading indicates the absence of Fn adsorption by the cellulose surface.

This observation is confirmed by the absence of positive labelling if, after having been incubated with serum, or with melanoma cells secreting Fn, the cellulose-coated dish is subjected to indirect immunolabelling (rabbit primary antibody reacting with the bovine Fn and murine Fn, goat anti-rabbit secondary antibody coupled with rhodamine). In the same situations, a positive result is obtained with the PS control.

3) Inducing Effect on Adherent Cells: Polystyrene (PS) Control for Cell Culture

The cells are cultured in medium supplemented by 10% foetal calf serum, 1% L-Glutamine and 0.5% antibiotics (penicillin, streptomycin).

—a—cell morphology: this is observed with a reverse phase-contrast microscope, or an environmental scanning electronic microscope (ESEM).

Rounded and aggregated cells (plated on PS): murine lines: Swiss 3T3, L929 fibroblasts, B16 C3, B16 F0, B16 F10 melanomas, MC 3T3 E1 osteoblasts subclones 4 and 24, primary metastasis cells of B16F10 melanomas induced in C57 Black 6 mice are used.

The aggregation of the B16 melanoma cells is dependent on calcium: in the absence of Ca⁺⁺ (medium without serum, monencin, tetrandrine), the percentage of cells isolated is very significantly increased for the 3 lines after 1 and 3 hours.

—b—proliferation: 10,000 cells are seeded per cm². The cells are counted (Malassez hemocytometer) after culture for 24 and 48 hours.

At 24 hours, any differences observed between the PS control and the cellulose coating are not significant.

On the other hand, at 48 hours, the cells on cellulose are two times less numerous than those on PS.

This was verified with all the lines studied.

It should be noted that the B16 F0 and B16 F10 lines proliferate more on cellulose that the B16 C3 line, which is non-metastatic. The number of B16 F0 and F10 cells is multiplied by 3 on cellulose. It is multiplied by 6 on PS, in the presence or absence of MSH 10⁻⁷ M melanin-stimulating hormone used as a second control. The number of B16 C3 cells remains stable after 48 hours on cellulose, it is multiplied by 2 on PS in the presence as in the absence of MSH.

Cultures in semi-solid medium of explants of metastases induced in mice by the injection of melanoma B16F10 cells (according to the technique described by Duval et al.: 1999. Cell & Materials, 9, 31-42) have shown, after 14 days in contact with BCD samples, very significant inhibition of the proliferation of the cells around the explant, compared with the cell vela largely developed in contact with the control PS samples.

—c—viability: trypan blue exclusion test

After culture for 48 hours, the viability of the round and aggregated cells on cellulose is less than that measured on PS (approximately 94% on cellulose and 99% on PS).

The viability of the MC3T3 E1 osteoblasts is greatly affected on BCD. 50% of the cells initially seeded have disappeared at 24 hours, and 75% at 48 hours. (cf. f-apoptosis).

—d—syntheses:

Total proteins per cell (assay by Bradford's method, commercial kit): the Swiss 3T3 and B16 lines exhibit increased synthesis after 48 hours on cellulose. The other lines have not been studied.

Fn synthesis: the cells aggregated on cellulose have positive peripheral labelling as from 3 hours, increased at 24 hours (indirect immunolabelling using a mouse anti-Fn primary antibody, which is non-reactive vis-a-vis bovine Fn). MSH also promotes an increase in Fn synthesis which appears well localized at the level of the cell contacts for the three melanoma lines cultured on PS.

Intracellular melanin synthesis: according to the technique described by De Pauw-Gillet et al. (Anticancer Research 1990, 10, 391-96).

A significant increase in intracellular melanin is observed after 48 hours for the 3 B16 lines aggregated on cellulose. The inducing effect of the cellulose coating on melanin synthesis is at least equivalent to if not greater than that obtained with MSH 10⁻⁷ M

This result is completed and confirmed by an increase in tyrosinase activity in the 3 lines cultured on cellulose at 48 hours. The assay method used is that described by Steinberg et al. (J Cell. Physiol. 1976, 87, 265-76). There too, the results are equivalent to or greater than those measured in the presence of MSH.

Cultures in semi-solid medium of explants of metastases induced in mice by the injection of B16F10 cells have shown, after 14 days in contact with BCD samples, a significant load of melanin in the cells around the explant, compared with amelanic cell vela largely developed in contact with the control PS samples.

—e—expression of membrane proteins: receptors and markers

By immunolabellings and western blotting, the N cadherins have been evaluated. The cells aggregated on cellulose show an increase in N cadherins. However, a reduction in β-catenins (immunoprecipitation and western blotting) suggests a loss of functionality of the N cadherins expressed on cellulose.

The Mel-Cam tumor marker, which can be observed starting with B16 F10 cells (western blots) cultured for 48 hours on PS is no longer expressed at the surface of the cells cultured at the same time on cellulose.

—f—apoptosis: determined by the measurement of caspase 3 activity by fluorometry or western blotting, and the TUNEL method (commercial kit).

The caspase 3 activity is generally increased at 24 and 48 hours in the cells aggregated on cellulose. This result is confirmed by the TUNEL test. The cells tested are: Swiss 3T3 fibroblasts, MC 3T3 E1 osteoblasts, and the 3 B16 melanoma lines. The other lines and the primary cultures of metastases induced by the injection of B16 F10 cells in mice are subsequently verified.

Complementary tests by labelling with Annexin V in flow cytometry have shown prolonged survival of the cells of B16 melanomas at 5 and 8 days and even beyond in the case of B16C3. On the other hand, the osteoblasts of the MC3T3 E1 line enter apoptosis very prematurely (before culture for 24 hours).

The BCDs therefore demonstrate different susceptibilities to apoptosis depending on the cell types analyzed.

Explants of metastases induced in mice by the injection of B16F10 cells, cultured for 14 days on semi-solid medium, have exhibited in contact with BCD samples a percentage of apoptotic cells which is significantly higher than that established starting with the controls cultured in contact with PS (methods used: Annexin V labelling in flow cytometry and TUNEL).

Conclusions

The inducing effect is a differentiating effect: cf. paragraphs b, d, and e, above.

The material has an effectiveness equal to or greater than MSH 10⁻⁷ M, a differentiating agent used as a control in cultures on PS.

The coating induces apoptosis in normal and cancerous cells: cf. paragraphs c, and f, above.

Prolonged culture test: 5 days, carried out with B16 C3.

Concluding results: very melanotic large clusters, maintenance of the aggregated state.

### BIBLIOGRAPHICAL REFERENCES

FAUCHEUX, N., WAROCQUIER-CLEROUT, R., HAYE, B., NAGEL, M-D.: 1998. “Cyclic AMP in cells adhering to bioincompatible (cuprophan) and biocompatible (AN 69) substrates.” J. Biomed. Mat. Res., 39, 506-510.
DUVAL, J-L., FAUCHEUX, N., WAROCQUIER-CLEROUT, R., NAGEL, M-D.: 1999. “Study of melanoma cell behavior in cell and organ cultures in vitro: use of biomaterials as potential tools of cell activation.” Cell & Materials, 9, 31-42.
FAUCHEUX, N., WAROCQUIER-CLEROUT, R., DUVAL, J-L., HAYE, B., NAGEL, M-D.: 1999. “cAMP levels in cells attached to AN 69 and Cuprophan: cAMP dependence of cell aggregation and the influence of serum.” Biomaterials, 20, 159-165.
FAUCHEUX, N., HAYE, B., NAGEL, M-D.: 2000. “Activation of cyclic AMP in cells adhering to biomaterials: regulation by vitronectin and fibronectin-integrin binding.” Biomaterials, 21, 1031-1038.
FAUCHEUX, N., DUFRESNE, M., NAGEL, M-D.: 2002. “Organisation of cyclic AMP-dependent connexin 43 in Swiss 3T3 cells attached to a cellulose substratum.” Biomaterials, 23, 413-421.
FAUCHEUX, N., CORREZE, C., HAYE, B., NAGEL, M-D.: 2001. “Accumulation of cyclic AMP in Swiss 3T3 cells adhering to a cellulose biomaterial substratum through interaction with adenylyl cyclase.” Biomaterials, 22, 2993-2998.
FAUCHEUX N., NAGEL M-D.: 2002. “Cyclic AMP—dependent aggregation of Swiss 3T3 cells on a cellulose substratum (Cuprophan) and decreased cell membrane Rho A.” Biomaterials, 23, 2295-2301.
FAUCHEUX N., ZAHM J M., BONNET N., LEGEAY G., NAGEL M-D.: 2004 “Gap junction communication between cells aggregated on a cellulose-coated polystyrene: influence of connexin 43 phosphorylation.” Biomaterials, 25, 2501-2506.
GEKAS J., HINDIE M., FAUCHEUX N., LANVIN O., MAZIERE C., FUENTES V., GOUILLEUX-GRUART V., DAVID B., MAZIERE J C., LASSOUED K., NAGEL MD.: 2004. “The inhibition of cell spreading on a cellulose substrate (Cuprophan) induces an apoptotic process via a mitochondria-dependent pathway.” FEBS Lett., 563, 103-107.

1-10. (canceled)

11. Bioactive dishes for cell cultures comprising on their bottom a bilayer comprising an internal primary layer made of hydroxypropylmethylcellulose (HPMC), or polyvinyl alcohol (PVA) in contact with the bottom of the dishes, and an external bioactive layer made of carboxypropylmethylcellulose situated on said internal layer.

12. Bioactive dishes according to claim 11, wherein the bioactive dishes are presented in the form of Petri dishes, such as polystyrene Petri dishes of commercial origin, or in the form of multi-well plates, on the bottom of which the bilayer is situated.

13. Bioactive dishes according to claim 11, wherein the thicknesses of the internal HPMC or PVA layer, and of the external CMC layer, are a few microns, in particular approximately 1 to 5 microns.

14. Method for preparing bioactive dishes according to claim 11, said method comprising:
a stage of activation of the surface of the bottom of the dishes by electromagnetic discharges,
depositing the internal HPMC layer on the bottom of the dishes, then drying, and
depositing the external bioactive layer on the dried primary layer obtained in the preceding stage, then drying.

15. Bioactive dishes according to claim 11, to carry out:
methods for studying cell ageing, cell differentiation, and apoptosis,
methods for screening anti-ageing molecules intended to prevent and delay the effects of ageing,
methods for screening antitumor molecules intended for the treatment of cancer,
methods for in vitro diagnosis of tumor-cell malignancy by measurement of the residual ability of cancer cells to differentiate, and to enter into apoptosis and therefore methods for in vitro tumor prognosis, and
study methods relating to research into signalling controlling morphology, bioadhesion, cell proliferation and intercellular communication.

16. Method for screening anti-ageing molecules intended to prevent and delay the effects of ageing, said method comprising:
a stage of culturing cells, such as fibroblasts, in the presence of the anti-ageing molecules to be studied, in the culture dishes defined in claim 11,
observing the cells by microscope in order to study their morphology, and/or the detection, or even the quantification, of the proliferation and syntheses, and
comparing the observations and results obtained on cultures of cells used as controls, said control cultures being carried out by culturing said cells in the dishes in the absence of said anti-ageing molecules to be studied.

17. Method for screening antitumor molecules intended for the treatment of cancer, comprising:
a stage of culturing cells, such as animal or human melanoma cells, in the presence of the antitumor molecules to be studied, in the culture dishes defined in claim 11,
observing the cells by microscope in order to study their morphology and their differentiation, and/or the detection, or even the quantification, of their proliferation, differentiation and apoptosis, and
comparing with the observations and results obtained on cell cultures used as controls, said control cultures being carried out by culturing said cells in the culture dishes in the absence of said antitumor molecules to be studied.

18. Method for in vitro diagnosis of the malignancy of tumor cells by measurement of the residual ability of cancer cells to differentiate, said method comprising:
a stage of culturing cancer cells, such as human melanoma cells obtained from biopsies, in the culture dishes defined in claim 11,
observing the cells by microscope in order to study their morphology and differentiation, and/or
detecting or quantifying, of their proliferation viability and apoptosis.

19. Application of the diagnosis method according to claim 18 to the in vitro prognosis of tumors.
